(19) United States
(12) Patent Application Publication
(10) Pub. No.: US 2009/0209989 A1
(43) Pub. Date: Aug. 20, 2009

(54) Micro-Flail Assembly And Method Of Use For The Preparation Of A Nucleus/Vertebral End Cap Of A Spine

(75) Inventors: Doris M. BLAKE, Delray Beach, FL (US); John B. SLEDGE, Marblehead, MA (US)

Correspondence Address: Clements Bernard PLLC, 1901 Roxborough Road, Suite 300, Charlotte, NC, 28211, US

(73) Assignee: U. S. SPINAL TECHNOLOGIES, L.L.C., Boca Raton, FL (US)

(21) Appl. No.: 12/370,964

(22) Filed: Feb. 13, 2009

Related U.S. Application Data
(60) Provisional application No. 61/028,329, filed on Feb. 13, 2008.

(51) Int. Cl. A61B 17/32

(52) U.S. Cl. 606/167

(57) ABSTRACT

The present disclosure provides a micro-flail assembly and associated method of use for the preparation of a nucleus/vertebral end cap of a spine. The micro-flail assembly is utilized in the formation of a nucleus/vertebral end cap between adjacent vertebrae while simultaneously protecting an adjacent annulus with a protective sheath. The protective sheath also acts as a guide while the micro-flail assembly is pivoted to form the end cap. Advantageously, the present invention can be utilized with a variety of surgical procedures including minimally invasive surgery. The formation of the end cap can be done in preparation of providing an insert device (e.g., bone graft, cage, artificial disc, or the like).

### CROSS-REFERENCE TO RELATED APPLICATION(S)

The present non-provisional patent application claims the benefit if priority to U.S. Provisional Patent Application Ser. No. 61/028,329, filed Feb. 13, 2008, and entitled “MICRO-FLAIL ASSEMBLY FOR THE PREPARATION OF A NUCLEUS/VERTEBRAL END CAP OF A SPINE,” the contents of which are incorporate in full reference herein.

### FIELD OF THE INVENTION

The present invention relates generally to spinal surgical devices and associated methods of use. More particularly, the present invention provides a micro-flail assembly and associated method of use for the preparation of a nucleus/vertebral end cap of a spine for receiving an insert device such as, for example, a bone graft, a cage, an artificial disc, and the like while simultaneously protecting an adjacent annulus during the formation of the end cap.

### BACKGROUND OF THE INVENTION

Various spinal surgical procedures and associated devices are conventionally implemented for spinal injuries such as interbody fusion and the like. These procedures and associated devices can include inserts placed between adjacent vertebrae. Inserts come in a variety of shapes and sizes and are made of a variety of materials. These inserts can be provided to promote fusion of the adjacent vertebrae such as bone grafts, cage devices, or other types of implants. Other inserts can also be used for a variety of purposes such as artificial spinal discs and the like.

With these spinal surgical procedures, there exists a need to prepare the nucleus/vertebral end cap or end plate of a spine. For example, a spinal disc that resides between adjacent vertebral bodies maintains spacing between the associated vertebral bodies and allows for relative motion between the vertebrae (in a healthy spine). A surgeon must prepare an opening at the site of the intended fusion or other insert by removing some or all of the disc material that exists between the adjacent vertebral bodies to be fused. Because the outermost layers of bone of a vertebral end plate are relatively inert to new bone growth, the surgeon must work on the end plate to remove at least the outermost cell layers of bone to gain access to the blood-rich, vascular bone tissue within the vertebral body. In this manner, the vertebrae are prepared in a way that encourages new bone to grow onto or through an insert that is placed between the vertebrae.

Conventional mechanisms of forming this space between adjacent vertebrae generally include: hand held biting and grasping instruments known as rongeurs; drills and drill guides; rotating burrs driven by a motor; and osteotomes and chisels. Sometimes the vertebral end plate must be sacrificed as occurs when a drill is used to drill across the disc space and deeper into the vertebrae than the thickness of the end plate. Such a surgical procedure necessarily results in the loss of the hardest and strongest bone tissue of the vertebrae—the end plate—and thereby robs the vertebrae of that portion of its structure best suited to absorbing and supporting the loads placed on the spine by everyday activity. Nevertheless, the surgeon must use one of the above instruments to work upon the adjacent end plates of the adjacent vertebrae to access the vascular, cancellous bone that is capable of participating in the fusion and causing active bone growth, and also to attempt to obtain an appropriately shaped surface in the vertebral bodies to receive the insert. Because the end plates of the adjacent vertebrae are not flat, but rather have a compound curved shape, and because the inserts, whether made of donor bone or a suitable implant material, tend to have a geometric rather than a biologic shape, it is necessary to conform the vertebrae to the shape of the insert to be received.

It is important in forming the space between the adjacent bone structures to provide a surface contour that closely matches the contour of the inserts so as to provide an adequate support surface across which the load transfer between the adjacent bone structures can be evenly applied. In instances where the surgeon has not been able to form the appropriately shaped space for receiving the inserts, those inserts may slip or be forcefully ejected from the space between the adjacent vertebrae, or lacking broad contact between the insert and the vertebrae, a failure to obtain fusion may occur.

Furthermore, conventional forming mechanisms are difficult to implement with minimally invasive surgery (MIS). Such MIS procedures are becoming the procedures of choice for spinal surgery. Thus there exists a need for a device and associated method of use that can form the nucleus/vertebral end cap of a spine while protecting adjacent material, such as the annulus.

### BRIEF SUMMARY OF THE INVENTION

In various exemplary embodiments, the present invention provides a micro-flail assembly and associated method of use for the preparation of a nucleus/vertebral end cap of a spine. The micro-flail assembly is utilized in the formation of a nucleus/vertebral end cap between adjacent vertebrae while simultaneously protecting an adjacent annulus with a protective sheath. The protective sheath also acts as a guide while the micro-flail assembly is pivoted to form the end cap. Advantageously, the present invention can be utilized with a variety of surgical procedures including minimally invasive surgery. The formation of the end cap can be done in preparation of providing an insert device (e.g., bone graft, cage, artificial disc, or the like).

In an exemplary embodiment of the present invention, a micro-flail assembly for forming a nucleus/vertebral end cap of a spine includes a cutting head; a protective sheath substantially encasing the cutting head on one side; a pivoting mechanism operable to pivot the cutting head; and an elongated outer sheath connected to the cutting head, the protective sheath, and the pivoting mechanism. The micro-flail assembly further includes a torque mechanism to rotate the cutting head. The torque mechanism can include a worm gear arrangement with a first shaft with a first worm gear, an idler gear rotatably engaged to the first worm gear, and a second shaft with a second worm gear rotatably engaged to the idler gear. The first shaft is disposed within the cutting head and the second shaft is disposed within the elongated outer sheath, and wherein the second shaft is adapted to receive a torque providing device to provide rotational force through the worm gear arrangement to the first shaft. The pivoting mechanism includes a guide rod disposed within the elongated outer shaft and adjacent to the first worm gear, and wherein movement on the guide rod translates to pivoting of the first shaft relative to the idler gear. Optionally, the pivoting mechanism is operable to pivot the cutting head up to 120 degrees. The first shaft can include a plurality of flails extending outward from the first shaft. Optionally, the plurality of flails each includes a barb disposed at an end of each of the plurality of flails. Alternatively, the cutting head includes a plurality of flails extending outward from a first shaft and a gearing arrangement rotatably connected to the torque mechanism. The protective sheath covers a portion of a front of the cutting head. Optionally, the micro-flail assembly is utilized in a minimally invasive surgical procedure. The elongated outer sheath can include an irrigation channel.

In another exemplary embodiment of the present invention, a method of forming a nucleus/vertebral end cap of a spine includes the steps of: inserting a device in a receiving patient; positioning the device relative to adjacent vertebrae; providing torque to the device to form the nucleus/vertebral end cap; pivoting the device while providing torque to the device to continue forming the nucleus/vertebral end cap; and protecting the annulus associated with the adjacent vertebrae while providing torque and pivoting the device with a protective sheath disposed to the device. The method further includes the step of: utilizing the protective sheath to guide the device while pivoting the device. The positioning the device step includes positioning the device relative to the adjacent vertebrae such that the protective sheath is positioned at one end of the nucleus/vertebral end cap with the protective sheath facing the adjacent annulus. The pivoting the device step includes rotating the device such that the protective sheath protects the adjacent annulus while the torque to the device forms the nucleus/vertebral end cap. Optionally, the device includes a cutting head, wherein the protective sheath substantially encases the cutting head on one side; a pivoting mechanism operable to pivot the cutting head; and an elongated outer sheath connected to the cutting head, the protective sheath, and the pivoting mechanism.

In yet another exemplary embodiment of the present invention, an apparatus for forming a nucleus/vertebral end cap of a spine includes a first shaft with a first worm gear at one end and adapted to receive torque at the other end; an idler gear rotatably connected to the first worm gear; a second shaft with a second worm gear at one end and disposed to an end of a protective sheath at the other end, wherein the second worm gear is rotatably and pivotably connected to the idler gear; a guide rod disposed to the second shaft at one end and encased in an outer sheath at the other end, wherein movement of the guide rod translates to pivoting of the first shaft relative to the idler gear; and a plurality of flails disposed to the first shaft and operable to rotate responsive to torque to thereby form the nucleus/vertebral end cap. The protective sheath substantially encases the plurality of flails on one side and a portion of a front of the first shaft thereby protecting adjacent annulus while forming the nucleus/vertebral end cap. Optionally, the apparatus is utilized in a minimally invasive surgical procedure.

### BRIEF DESCRIPTION OF THE DRAWINGS

The present invention is illustrated and described herein with reference to the various drawings, in which like reference numbers denote like method steps and/or system components, respectively, and in which:

FIG. 1 is a top view of a micro-flail assembly for the preparation of a nucleus/vertebral end cap of a spine according to an exemplary embodiment of the present invention;

FIG. 2 is a perspective view of the worm gear arrangement for the micro-flail assembly of FIG. 1 according to an exemplary embodiment of the present invention;

FIG. 3 is a perspective view of the sheath and the flail shaft for the micro-flail assembly of FIG. 1 according to an exemplary embodiment of the present invention;

FIG. 4 is a front view of the sheath and the one or more flails for the micro-flail assembly of FIG. 1 according to an exemplary embodiment of the present invention;

FIG. 5 is a top view of the micro-flail assembly of FIG. 1 engaging a nucleus/vertebral end cap according to an exemplary embodiment of the present invention;

FIG. 6 is a side of vertebrae with the micro-flail assembly of FIG. 1 according to an exemplary embodiment of the present invention;

FIG. 7 is a front of vertebrae with the micro-flail assembly of FIG. 1 according to an exemplary embodiment of the present invention; and

FIG. 8 is a flowchart of a method of use associated with the micro-flail assembly of FIG. 1 according to an exemplary embodiment of the present invention.

### DETAILED DESCRIPTION OF THE INVENTION

In various exemplary embodiments, the present invention provides a micro-flail assembly and associated method of use for the preparation of a nucleus/vertebral end cap of a spine. The micro-flail assembly is utilized in the formation of a nucleus/vertebral end cap between adjacent vertebrae while simultaneously protecting an adjacent annulus with a protective sheath. The protective sheath also acts as a guide while the micro-flail assembly is pivoted to form the end cap. Advantageously, the present invention can be utilized with a variety of surgical procedures including minimally invasive surgery (MIS). The formation of the end cap can be done in preparation of providing an insert device (e.g., bone graft, cage, artificial disc, or the like).

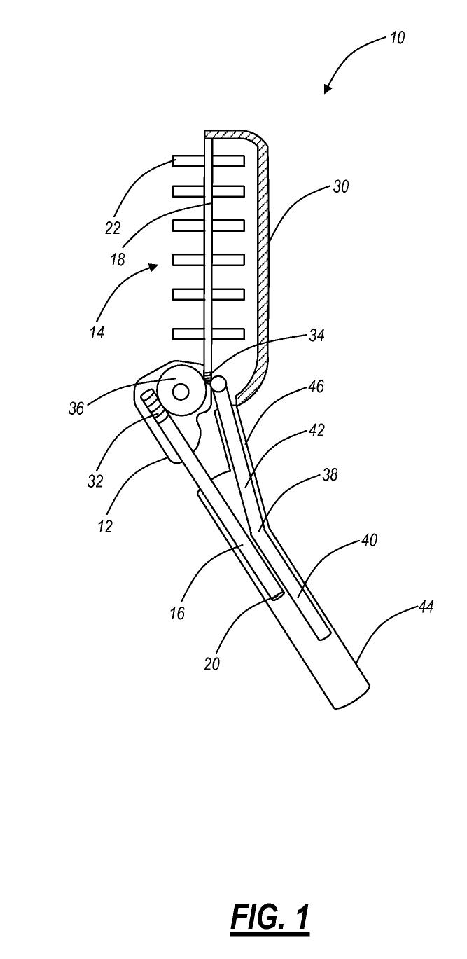

Referring to FIG. 1, a top view illustrates a micro-flail assembly 10 for the preparation of a nucleus/vertebral end cap of a spine according to an exemplary embodiment of the present invention. The micro-flail assembly 10 includes a live head 12 interconnected to a dead head 14 through a worm gear arrangement configured to transfer torque from a drive shaft 16 to a flail shaft 18. The drive shaft 18 can receive torque through various mechanisms such as a drill or the like (not shown) attached to one end 20 of the drive shaft 16. The dead head 14 is configured to operate as a pivotable cutting head relative to the live head 12.

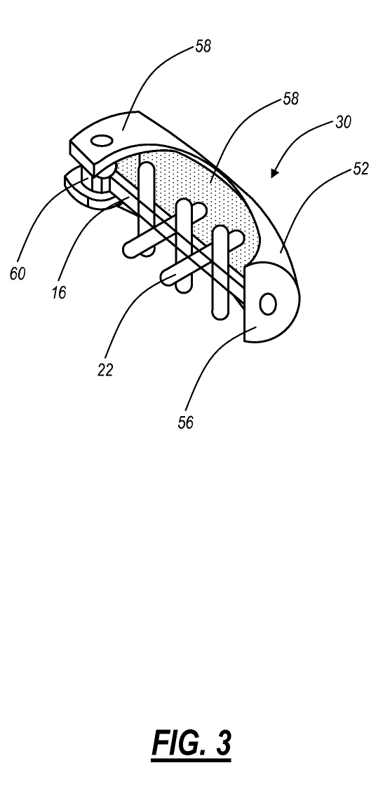

The flail shaft 18 includes one or more flails 22 that are disposed or connected to the flail shaft 18, i.e., the flail shaft 18 includes a plurality of flails (the one or more flails 22) extending outward from the flail shaft 18. In the exemplary embodiments described herein, the one or more flails 22 are illustrated forming right angles between adjacent flails 22. The present invention also contemplates other arrangements of the one or more flails 22. The one or more flails 22 rotate responsive to torque in the worm gear arrangement thereby forming a nucleus/vertebral end cap of a spine, i.e. cutting the end cap to a shape as required for an appropriate insert. Optionally, the one or more flails 22 can include barbs at the end for improved cutting (as illustrated in FIG. 3). Alternatively, the present invention contemplates additional cutting mechanisms in lieu of the flails 22 as are known in the art. The flail shaft 18 is connected to a sheath 30 (illustrated in FIG. 1 in a cross-sectional view). The sheath 30 is operable to protect annulus and it operates a guide to follow the annulus and keep the flail assembly 10 in a nucleus of the spine. The sheath 30 is illustrated in a cross-sectional view in FIG. 3.

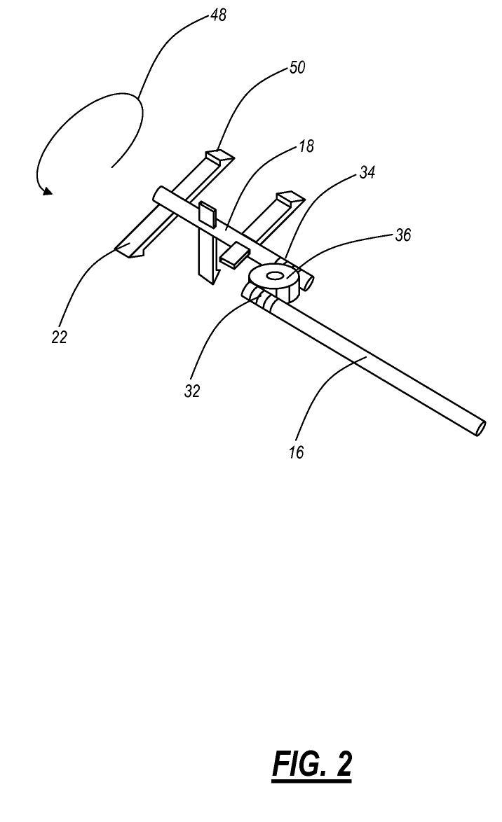

The worm gear arrangement includes a worm 32 on the drive shaft 16 and a worm 34 on the flail shaft 18 with the worms 32, 34 interconnected through an idler gear 36. The worm gear arrangement is illustrated in FIG. 2. The worm 32 is connected or attached to the drive shaft 16 and rotatably engages the idler gear 36 responsive to torque on the drive shaft 16. Accordingly, rotation on the idler gear 36 causes the worm 34 to rotate. The worm 34 is connected or attached to the flail shaft 18. Thus torque in the drive shaft 16 is translated to torque on the flail shaft 18 thereby causing rotation/vibration of the one or more flails 22. Those of ordinary skill in the art will recognize the worm gear arrangement is shown for illustration purposes and the present invention contemplates other mechanisms of providing torque to the one or more flails 22 as is known in the art.

The dead head 14 on the micro-flail assembly 10 is configured to pivot with respect to the live head 12. This enables a surgeon to position the micro-flail assembly 10 at a vertebral body and to rotate the dead head 14 with the one or more flails 22 to form the associated end cap. The flail shaft 18, the one more flails 22, and the protective sheath all pivot with the dead head 14. The micro-flail assembly 10 includes a guide rod 38 which is disposed or connected to the flail shaft 18 for pivoting the dead head 14 in relation to the live head 12. The guide rod 38 includes a straight portion 40 and an angled portion 42. The straight portion 40 is included and terminates in an outer sheath 44 that also includes the drive shaft. The angled portion 42 is adjacent to the worm 34 on the flail shaft 18. Movement of the guide rod 38, such as from a surgeon, translates to rotation of the flail shaft 18 relative to the idler gear 36 thus causing pivoting of the entire dead head 14. This pivoting while torque is provided to the device results in a curling motion that forms the end cap while protecting the exterior of the end cap, i.e. the annulus. For example, the outer sheath 44 can include a handle portion or the like (not shown) for the surgeon to operate the micro-flail assembly 10 and move the guide rod 38. The outer sheath 44 can include a portion for receiving a torque generating device to engage the drive shaft 16 and a portion for rotating or manipulating the guide rod 38 to pivot the dead head 14.

Additionally, the outer sheath 44 can include an irrigation sheath/channel 46 which encases the angled portion 42 of the guide rod 38 and which is disposed to the sheath 30. The irrigation sheath/channel 46 provides for removal of material that is formed by the one or more flails 22 as well as for providing irrigation or the like to the vertebrae during forming. The various components described herein with respect to the micro-flail assembly 10 can be manufactured from a metal or another biocompatible material.

Referring to FIG. 2, a perspective view of the worm gear arrangement is illustrated for the micro-flail assembly 10 according to an exemplary embodiment of the present invention. As described herein, the idler gear 36 translates torque from the drive shaft 16 through the worm gear 32 to the flail shaft 18 through the worm gear 34. The resulting torque causes the flail shaft 18 to rotate (in direction 48). Also, the gears 32, 34, 36 can be configured to alternate directions to cause the flail shaft 18 to vibrate or rotate back and forth. Optionally, the one or more flails 22 disposed to the flail shaft 18 can include barbs 50 to assist in forming the end cap.

Referring to FIG. 3, a perspective view of the sheath 30 and the flail shaft 18 is illustrated for the micro-flail assembly 10 according to an exemplary embodiment of the present invention. The sheath 30 is operable to protect an annulus associated with a vertebral body. This is accomplished by covering a portion of the front and substantially all of one side of the flail shaft 18 and the one or more flails 22. In operation, the sheath 30 is positioned such that the one or more flails face towards a center of the vertebral body thereby protecting the annulus. With the pivoting motion of the sheath 30 and the other components, the exterior part, i.e. the annulus, avoids damage during the formation of the end cap.

The sheath 30 includes a curved exterior body 52, an interior 54, a front 56, and a back 58. The curved exterior body 52 is shaped to assist in guiding the sheath 30 and therefore the micro-flail assembly 10 to follow the annulus as well as protecting the annulus and keeping the sheath 30 in the nucleus. The curved exterior body 52 also prevents the annulus from being damaged while the sheath 30 and the rest of the dead head 14 are pivoted within an end cap. The one or more flails 22 are able to rotate and/or vibrate freely, i.e. the interior 54 is positioned to enable clearance of each of the one or more flails 22 and to prevent the one or more flails 22 from contacting the annulus. The flail shaft 18 can be fixedly engaged to the front 56 and the back 58 of the sheath 30. The front 56 of the sheath 30 can also provide protection as well as providing guidance of the sheath 30 in the nucleus. The back 58 includes a notch 60 on the flail shaft 18. The notch 60 is operable to engage the guide rod 38 to pivot the sheath 30 and the associated components protected by the sheath 30.

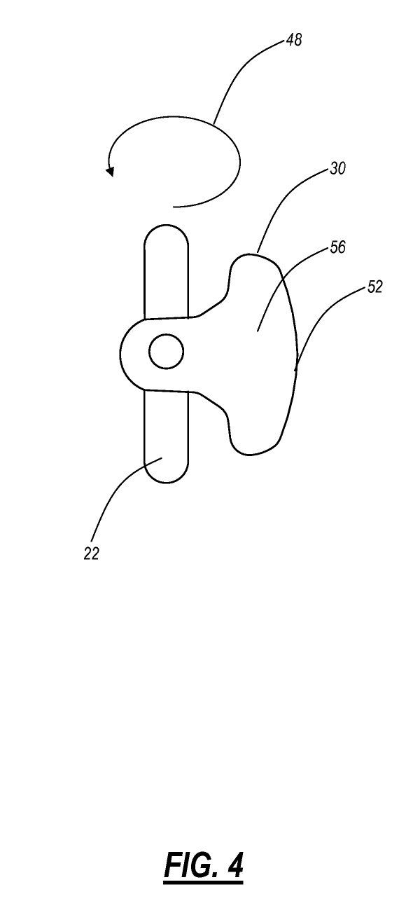

Referring to FIG. 4, a front view of the sheath 30 and one or more flails 22 is illustrated for the micro-flail assembly 10 according to an exemplary embodiment of the present invention. FIG. 4 illustrates a front view of the sheath 30 with a deployed flail 22. The flails 22 are configured to rotate and/or vibrate along a direction 48 to form an end cap of a nucleus/vertebral. The curved exterior body 52 protects an outside area from the movement of the flails 22, i.e. the annulus while the nucleus portion of a vertebral body is formed. Also of note, the compact structure and protective nature of the sheath 30 allow the micro-flail assembly 10 to be used in minimally invasive surgery (MIS) with the front 56 and the curved exterior body 52 allowing the dead head 14 to be inserted into a minimal incision and guided towards the vertebral body.

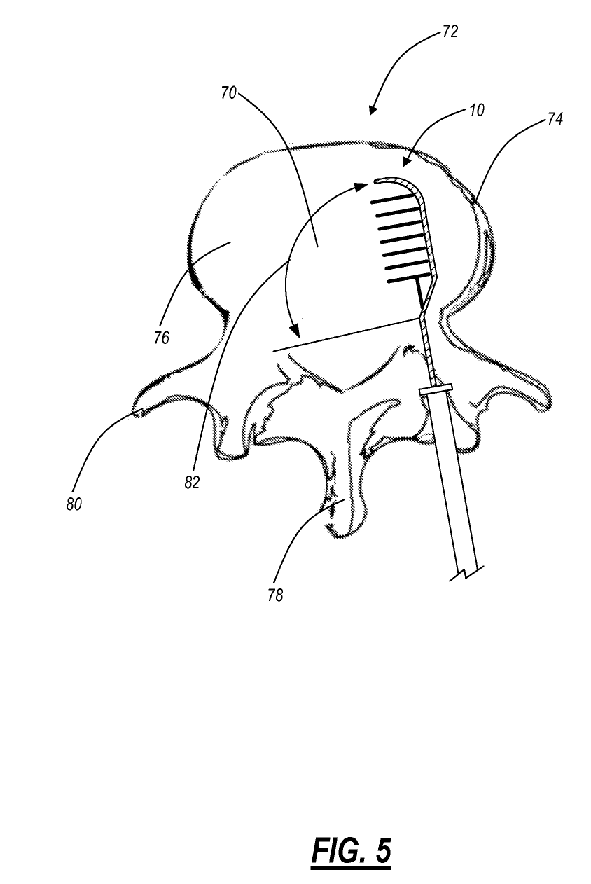

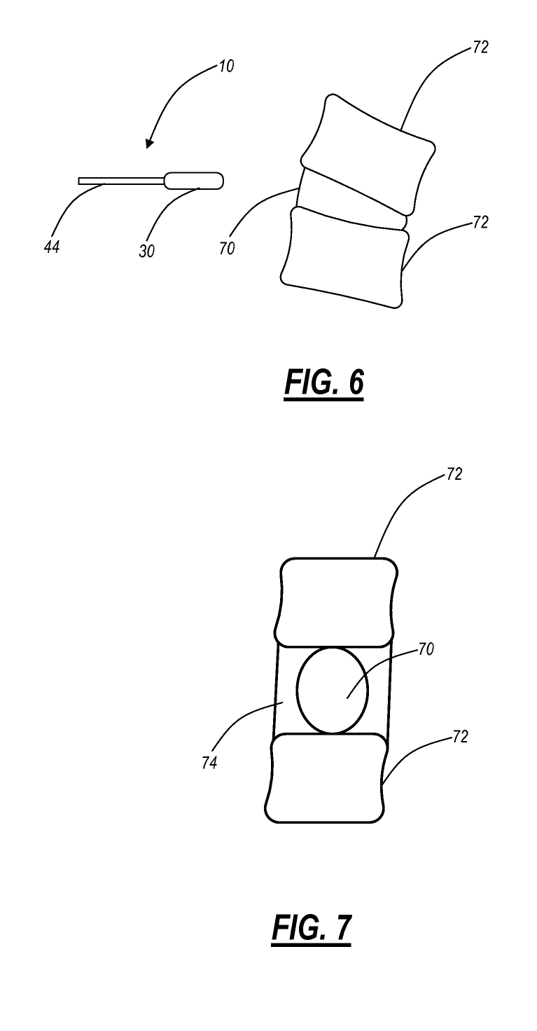

Referring to FIG. 5, a top view of the micro-flail assembly 10 is illustrated engaging a nucleus/vertebral end cap 70 of a vertebra 72 according to an exemplary embodiment of the present invention. The vertebra 72 includes an annulus 74, a nucleus 76, spinous processes 78, and transverse processes 80. FIG. 5 illustrates a top cross-sectional view of the vertebra (note, another vertebra is located on top of the vertebra 70 as illustrated in FIGS. 6 and 7). As described herein, the micro-flail assembly 10 is configured to form the nucleus/vertebral end cap 70 for receiving an insert (e.g., bone graft, cage, artificial disc, etc.). A surgeon can position the micro-flail assembly 10 between the adjacent vertebrae 72 and use the flails 22 to form the end cap 72. In this process, the sheath 30 is operable to protect the annulus 74 from the flails 22. Additionally, the dead head 14 of the micro-flail assembly 10 can pivot (e.g., up to 120 degrees and indicated, e.g., by a range of motion 82) through movement or the like of the guide rod 38. Advantageously, this enables the surgeon to form the end cap 70 with very little movement of the micro-flail assembly 10 while simultaneously protecting the annulus 74 from damage.

Referring to FIGS. 6 and 7, a side and front view of vertebrae 90 are illustrated with the micro-flail assembly 10 according to an exemplary embodiment of the present invention. As shown in FIG. 6, the micro-flail assembly 10 is inserted in a patient through a surgical technique. The sheath 30 provides protection for the receiving patient to prevent contact with the one or more flails 22 during insertion, during operation, and during removal. The sheath 30 with the one or more flails 22 is positioned between the adjacent vertebra 72 to form the nucleus/vertebral end cap 70. The micro-flail assembly 10 is positioned in area of the nucleus/vertebral end cap 70 and the one or more flails 22 are engaged with the sheath protecting the adjacent annulus 74 while the nucleus/vertebral end cap 70 is formed with the one or more flails 22.

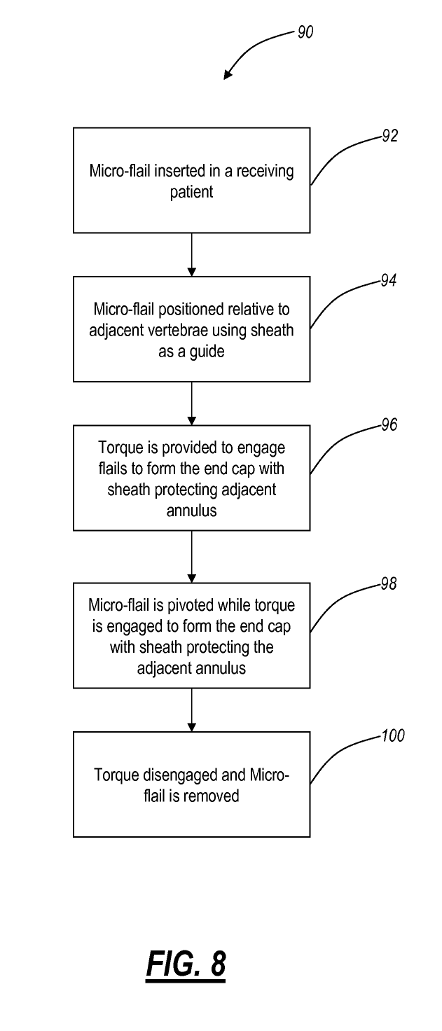

Referring to FIG. 8, a flowchart illustrates a method of use associated with the micro-flail assembly 10 according to an exemplary embodiment of the present invention. First, a micro-flail assembly is inserted in a receiving patient (step 92). The insertion can be done through any spinal surgical technique and the present invention contemplates compatibility with the various techniques used (posterior, lateral, anterior, etc.). Of note, the present invention is compatible with minimally invasive surgical techniques. For example, the sheath 30 can provide protection during insertion and removal as well as during operation of the micro-flail assembly.

The micro-flail is positioned relative to adjacent vertebrae using the sheath 30 as a guide (step 94). Of note, the curved exterior surface of the sheath can provide an ability to maneuver the micro-flail within the receiving patient. Here, the micro-flail is positioned to engage the nucleus between the adjacent vertebrae. Once positioned, torque is provided to engage the flails to enable forming of the end cap with the sheath providing protection to the adjacent annulus (step 96). The torque can be provided by a variety of mechanisms known in the art such as, for example, a drill operably connected to the flails through a gearing arrangement or the like.

The micro-flail is pivoted while the torque is engaged to form the end cap between the adjacent vertebrae while the sheath simultaneously protects the adjacent annulus (step 98). For example, the micro-flail is positioned at one end of the end cap with the sheath facing the adjacent annulus. The flails are configured to form the portion of the end cap opposite of the adjacent annulus. The pivoting motion allows the micro-flail to form the interior of the end cap from the one end of the end cap while simultaneously avoiding the annulus due to the sheath. This pivoting enables formation of the end cap without requiring a surgeon to maneuver the micro-flail in the space. This is advantageous for MIS procedures. Finally, the torque is disengaged and the micro-flail is removed from the receiving patient (step 100). Once formed, the end cap can receive an insert or the like.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention and are intended to be covered by the following claims.

What is claimed is:

1. A micro-flail assembly for forming a nucleus/vertebral end cap of a spine, comprising:
a cutting head;
a protective sheath substantially encasing the cutting head on one side;
a pivoting mechanism operable to pivot the cutting head; and
an elongated outer sheath connected to the cutting head, the protective sheath, and the pivoting mechanism.

2. The micro-flail assembly of claim 1, further comprising:
a torque mechanism to rotate the cutting head.

3. The micro-flail assembly of claim 2, wherein the torque mechanism comprises a worm gear arrangement comprising a first shaft comprising a first worm gear, an idler gear rotatably engaged to the first worm gear, and a second shaft comprising a second worm gear rotatably engaged to the idler gear.

4. The micro-flail assembly of claim 3, wherein the first shaft is disposed within the cutting head and the second shaft is disposed within the elongated outer sheath, and wherein the second shaft is adapted to receive a torque providing device to provide rotational force through the worm gear arrangement to the first shaft.

5. The micro-flail assembly of claim 4, wherein the pivoting mechanism comprises a guide rod disposed within the elongated outer shaft and adjacent to the first worm gear, and wherein movement on the guide rod translates to pivoting of the first shaft relative to the idler gear.

6. The micro-flail assembly of claim 5, wherein the pivoting mechanism is operable to pivot the cutting head up to 120 degrees.

7. The micro-flail assembly of claim 4, wherein the first shaft comprises a plurality of flails extending outward from the first shaft.

8. The micro-flail assembly of claim 7, wherein the plurality of flails each comprises a barb disposed at an end of each of the plurality of flails.

9. The micro-flail assembly of claim 2, wherein the cutting head comprises a plurality of flails extending outward from a first shaft and a gearing arrangement rotatably connected to the torque mechanism.

10. The micro-flail assembly of claim 1, wherein the protective sheath covers a portion of a front of the cutting head.

11. The micro-flail assembly of claim 1, wherein the micro-flail assembly is utilized in a minimally invasive surgical procedure.

12. The micro-flail assembly of claim 1, wherein the elongated outer sheath comprises an irrigation channel.

13. A method of forming a nucleus/vertebral end cap of a spine, comprising the steps of:
inserting a device in a receiving patient;
positioning the device relative to adjacent vertebrae;
providing torque to the device to form the nucleus/vertebral end cap;
pivoting the device while providing torque to the device to continue forming the nucleus/vertebral end cap; and
protecting the annulus associated with the adjacent vertebrae while providing torque and pivoting the device with a protective sheath disposed to the device.

14. The method of claim 13, further comprising the step of:
utilizing the protective sheath to guide the device while pivoting the device.

15. The method of claim 13, wherein the positioning the device step comprises:
positioning the device relative to the adjacent vertebrae such that the protective sheath is positioned at one end of the nucleus/vertebral end cap with the protective sheath facing the adjacent annulus.

16. The method of claim 15, wherein the pivoting the device step comprises:
rotating the device such that the protective sheath protects the adjacent annulus while the torque to the device forms the nucleus/vertebral end cap.

17. The method of claim 13, wherein the device comprises:
a cutting head, wherein the protective sheath substantially encases the cutting head on one side;
a pivoting mechanism operable to pivot the cutting head; and
an elongated outer sheath connected to the cutting head, the protective sheath, and the pivoting mechanism.

18. An apparatus for forming a nucleus/vertebral end cap of a spine, comprising:
a first shaft comprising a first worm gear at one end and adapted to receive torque at the other end;
an idler gear rotatably connected to the first worm gear;
a second shaft comprising a second worm gear at one end and disposed to an end of a protective sheath at the other end, wherein the second worm gear is rotatably and pivotably connected to the idler gear;
a guide rod disposed to the second shaft at one end and encased in an outer sheath at the other end, wherein movement of the guide rod translates to pivoting of the first shaft relative to the idler gear; and
a plurality of flails disposed to the first shaft and operable to rotate responsive to torque to thereby form the nucleus/vertebral end cap.

19. The apparatus of claim 18, wherein the protective sheath substantially encases the plurality of flails on one side and a portion of a front of the first shaft thereby protecting adjacent annulus while forming the nucleus/vertebral end cap.

20. The apparatus of claim 18, wherein the apparatus is utilized in a minimally invasive surgical procedure.
